(19) United States
(12) Patent Application Publication
(10) Pub. No.: US 2018/0008830 A1
(43) Pub. Date: Jan. 11, 2018

(54) APPARATUS AND METHODS FOR OPTIMIZING INTRA-CARDIAC PRESSURES FOR IMPROVED EXERCISE CAPACITY

(71) Applicant: CardioFlow Technologies, LLC, Nashville, TN (US)

(72) Inventor: Daniel Walter Kaiser, Palo Alto, CA (US)

(21) Appl. No.: 15/655,809

(22) Filed: Jul. 20, 2017

Related U.S. Application Data
(63) Continuation-in-part of application No. 15/168,204, filed on May 30, 2016.
Application No. 15/168,204 is a continuation-in-part of application No. 14/597,190, filed on Jan. 14, 2015.
(60) Provisional application No. 62/364,663, filed on Jul. 20, 2016.
(60) Provisional application No. 62/168,784, filed on May 30, 2015.
(60) Provisional application No. 61/927,038, filed on Jan. 14, 2014.

(51) Int. Cl. A61N 1/365; A61N 1/05; A61B 5/00; A61N 1/368; A61B 5/0215; A61N 1/372; A61B 5/11; A61M 1/10; A61M 1/12

(52) U.S. Cl. CPC A61N 1/36585; A61N 1/37217; A61N 1/056; A61B 5/7278; A61B 5/686; A61B 5/1118; A61N 1/36842; A61B 5/0215; A61M 1/122; A61M 1/1074

(57) ABSTRACT

Systems and methods are provided for optimizing hemodynamics within a patient's heart, e.g., to improve the patient's exercise capacity. In one embodiment, a system is configured to be implanted in a patient's body to monitor and/or treat the patient that includes at least one sensor configured to provide sensor data that corresponds to a blood pressure within or near the patient's heart; at least one component designed to cause dyssynchrony of the right ventricle, and a controller configured for adjusting the function of the at least one component based at least in part on sensor data from the at least one sensor.

### RELATED APPLICATION DATA

The present application claims benefit of co-pending provisional application Ser. No. 62/364,663, filed Jul. 20, 2016, and is a continuation-in-part of co-pending U.S. application Ser. No. 15/168,204, filed May 30, 2016, which claims benefit of provisional application Ser. No. 62/168,784, filed May 30, 2015, and is a continuation-in-part of co-pending application Ser. No. 14/597,190, filed Jan. 14, 2015, which claims benefit of provisional application Ser. No. 61/927,038, filed Jan. 14, 2014, the entire disclosures of which are expressly incorporated by reference herein.

### FIELD OF THE INVENTION

The present invention relates to apparatus, systems, and methods to optimize hemodynamics within a patient's heart, e.g., in order to improve the patient's capacity for exercise and/or other physical activity.

### BACKGROUND

In healthy young individuals, increasing left-sided filling pressures within the individual's heart is associated with a proportional increase in left ventricular stroke volume; and therefore cardiac output. However, with age, the left ventricle becomes stiff. This process, often accelerated with decades of hypertension or diabetes, results in a failure of increased pressure to increase stroke volume. This pressure-volume relationship is also known as the Frank-Starling curve. Over time, the slope of this curve becomes flat. When this occurs, increasing left-sided filling pressures do not improve stroke volume (or do so only marginally). However, elevated left-sided filling pressures contribute to shortness of breath and fatigue. By preventing the right ventricle from causing excessive elevations in left-sided filling pressures, patients can have improved quality of life and improvement in exercise capacity.

### SUMMARY

The present invention is directed to an apparatus, systems, and methods to optimize hemodynamics within a patient's heart, e.g., in order to improve the patient's capacity for exercise and/or other physical activity. More particularly, the present invention is directed to implantable devices that optimize intra-cardiac filling pressures, and to systems and methods for using such devices.

Left sided filling pressures cannot increase unless the right ventricle transiently pumps more blood than the left ventricle. It is common for the left ventricle to develop dysfunction at a faster rate than the right ventricle. The result is that the right ventricle is able to pump more blood than the left ventricle, particularly with exercise. This imbalance of biventricular function results in rising left-sided filling pressures that back up into the pulmonary vasculature. The elevated pulmonary pressures contribute to increased respiratory rate and shortness of breath. Given this problem, systems and methods may be provided that attempt to balance biventricular function to prevent pressure-overload within the heart without compromising cardiac output. By impairing the function of the right ventricle, pressures may be reduced and/or cardiac output may even paradoxically increase. Since the cardiac output may increase, “impairing” may be a misnomer. The goal is to affect right ventricular function so as to re-balance biventricular function. Therefore, various methods may be used with the systems herein, e.g., creating a pressure gradient to blood flow (e.g., in the inferior vena cava), creating valvular dysfunction (such as tricuspid regurgitation), and/or impairing normal function of the right ventricle (such as changing the electrical activation from apex to base to prematurely activating the free wall of the right ventricle).

In one embodiment, the system includes methods to decrease right ventricular stroke volume through electrical stimulation. For example, the right ventricle is a triangular structure. Both the inflow and outflow of blood entering and leaving the right ventricle occurs at the base of the heart (the region of the right ventricle immediately adjacent the tricuspid valve and opposite the apex). Concordantly, the electrical system of the heart includes specialized conduction fibers such that the apex of the heart is stimulated first. Electrical conduction begins at the apex and propagates to the base, squeezing blood towards the base as the contraction wavefront travels from apex to base. If the electrical activation and subsequent mechanical function of the right ventricle were changed, the function of the right ventricle would be compromised. Therefore, the system may alter the timing of myocardial activation, e.g., in order to optimize right ventricular systolic, pulmonary and/or left-ventricular filling pressures.

In one embodiment, the system monitors pressures within the right ventricle, right atrium, pulmonary artery, left atrium, or coronary sinus. When these pressures are estimated to be elevated or rapidly rising, the system may deliver electrical impulses in order to temporarily alter the typical contraction pattern of the right ventricle. For example, in one embodiment, the system may stimulate myocardial tissue near or along the free wall of the right ventricle (i.e., the outer wall away from the left side of the heart) near the base of the heart. Stimulating this location may cause a contraction wavefront beginning from the base and extending to the apex, decreasing the total function of the right ventricle. Since this location is distant from the left ventricle, the contraction wavefront of the left ventricle is minimally or not affected. In some embodiments, the system includes one of more pacing electrodes in order to stimulate left ventricular myocardium. Therefore, this system of prematurely activating the right ventricle may be incorporated into devices similar to single chamber pacemaker systems, dual chamber pacemaker systems, and bi-ventricular pacing systems. By determining the precise timing of myocardial activation between the two ventricles, pulmonary pressures may be controlled or at least reduced.

In another embodiment, the pacing electrode may be connected to an elongate member. In other embodiments, the pacing electrode communicates to the rest of the device through wireless communication. The system may include multiple pacing mechanisms, such as an electrode at the right ventricular apex, multiple pacing electrodes in the coronary sinus in order to stimulate the left ventricle, as well as one or more electrodes for stimulating the base of the right ventricle. In one embodiment, a plurality of electrodes may be provided spaced apart from one another along a distal portion of the elongate member.

In other systems and methods, one or more of the electrodes may be wireless, implanted independently in different positions along the right ventricle. Such electrode devices may include a wireless communication interface, battery or other power source, and a processor for communicating with a remote controller, e.g., for activation instructions and the like, within a housing that may be secured or otherwise implanted within the right ventricle.

For example, one or more electrodes may be located near the base of the free wall of the right ventricle. When the one or more electrodes stimulate the myocardium, the right ventricle is activated at the base of the free wall and the muscle propagation travels towards the apex. This activation pattern prevents the right ventricle from pumping blood out of the right ventricle in an effective manner. In addition, there may be other electrodes located along the free wall but located closer to the apex of the heart. These electrodes may also result in an abnormal right ventricular activation pattern, however, not as severe as the electrodes located at the base. Therefore, the amount of right ventricular impairment may be changed based on timing and location of the electrodes that are controlled to stimulate the heart. Such changes may be used temporarily to decrease the stroke volume and/or otherwise modify the function of the right ventricle.

Optionally, while stimulating the right ventricle to decrease the normal function of the right ventricle, one or more additional actions may be taken. For example, if the system includes one or more pacing electrodes for pacing the heart, the rate of pacing may be increased to increase cardiac output and prevent volume overload. In particular, if the right ventricle is stimulated for an extended period of time, decrease in function of the right ventricle may result in increased total body blood volume and/or increased water absorption by the kidneys.

In addition or alternatively, if the system determines that the patient's pressures are elevated at rest, the system may activate an alarm and/or otherwise notify the patient and/or their caregiver. For example, such notice may be used to notify the caregiver to modify one or more medications being used to treat the patient.

The system may also determine the timing of right ventricular stimulation by sensing atrial activity. For example, the device may be programmed to sense the typical timing of ventricular activation. Based on this timing, the device may then determine the timing of right ventricular activation without requiring dedicated electrodes to the right ventricular apex, left ventricle, or coronary sinus electrodes. The device measures the normal timing of the local activation after either atrial pacing or atrial sensing. The timing of apical activation may be measured by far-field morphology analyses or may be programmed externally.

The timing of right ventricular free wall activation may be activated in such a way that the entire right ventricular activation occurs starting from the pacing electrode. Alternatively, the right ventricular electrode may be activated such that the paced wavefront fuses with the wavefront from the normal conduction pathway. By varying the prematurity of the right ventricular pacing compared to the normal timing of activation, the amount of right ventricular impairment may be controlled and titrated. Therefore, the degree of prematurity may be based on one or more of activity level, respiratory rate, blood pressure measurement, and/or other sensed parameter and further optimized based on physiological responses.

In one embodiment, the prematurity of right ventricular pacing is based on activity sensors, such as accelerometers or breathing rate sensors. Similar to current pacing devices, the rate of atrial pacing is determined by sensed activity level (which is usually obtained from accelerometer data). The more activity, the faster the atrial pacing rate. Similarly, the amount of right ventricular premature pacing may be based on activity level. That is, since pulmonary pressures and left ventricular filling pressures tend to be higher with higher activity/exertion levels, by delivering more premature right ventricular stimulation based on level of activity, the amount of pressure elevations may be minimized. The relationship between activity amount and premature pacing may be programmed into the device. In another embodiments, the respiratory rate at a given exertion or activity level is used as a guide to optimize the relationship between sensed activity and premature pacing.

In other embodiments, a pressure sensor is utilized to guide the amount of premature pacing. A pressure sensor may also determine which electrode to stimulate. In some cases, premature right ventricular pacing may cause cardiac ectopy within heart. Therefore, the device may need to monitor for cardiac ectopy, which may be caused by the abnormal right ventricular contraction, and reduce the prematurity of the pacing to reduce the ectopic beats.

In another embodiment, the system may decrease the stroke volume of the right ventricle. During exercise, the increase in right ventricular filling pressures frequently dilates the right ventricle diastolic volume and increase the stroke volume. However, the left ventricle, which frequently has significantly more diastolic dysfunction, often cannot increase the stroke volume similarly, ultimately resulting in elevations in pulmonary and left-sided filling pressures. By preventing the right ventricle stroke volume from increasing beyond what is tolerated by the left ventricle, exercise capacity and patient symptoms may be improved.

In one embodiment, a mechanical, adjustable component is placed within the right ventricle to decrease the stroke volume. This component may provide a communication to the pulmonary artery and/or right atrium by a connection across the pulmonary and/or tricuspid annulus. The volume of the component within the right ventricle may therefore be adjusted to control the stroke volume of the right ventricle. In another embodiment, the component may be placed along the free wall of the right ventricle, and prevent the right ventricle from enlarging in response to elevations in filling pressures. In this and similar embodiments, a component could be placed outside of the right ventricular myocardium within the pericardial sac, in order to influence right ventricular function. The adjustable component may also simply enlarge within the right ventricle to decrease stroke volume.

In another embodiment, like a typical pacemaker, the systems and methods herein may pace a patient's heart according to patient activity. In addition, the systems may increase the lower rate limit in response to an increase in total body blood volume. For example, when the system is actively attempting to reduce filling pressures when the patient is at rest, the system may increase the lower rate limit of the device. By increasing the pacing rate, the cardiac output may increase and encourage diuresis. Therefore, if the system senses that the patient is at rest, however, the system is attempting to reduce filling pressures, the system may increase the lower rate limit of the system. Since many patients have an imbalance between biventricular function, increasing the pacing rate may increase left-sided filling pressures; and therefore the system may simultaneously respond to rising filling pressures as the pacing rate is increased. Therefore, increased patient activity may result in increasing the patient's heart rate. Furthermore, if the system is attempting to reduce filling pressures at rest, this would also increase the patient's heart rate to increase the cardiac output.

In accordance with another embodiment, a system is provided that is configured to be implanted in a patient's body to monitor and/or adjust the electrical system of the patient's heart, the system including at least one sensor acquiring signals corresponding to patient activity or movement; at least one pacing component positioned adjacent a free wall of a right ventricle of the heart, whereby stimulation of the at least one pacing component is designed to compromise function of the right ventricle; and a controller coupled to the at least one sensor and at least one pacing component for adjusting the function of the at least one pacing component based at least in part on the signals from the at least one sensor.

In accordance with still another embodiment, a system is provided for monitoring and/or treating a heart of a patient to increase the patient's capacity for physical activity that includes a blood pressure sensor implantable within a region of the heart; an adjustable component configured to affect contractility of a right ventricle of the heart; an activity sensor implantable within the patient's body; at least one pacing component sized for introduction into a region of the heart; and a processor operatively coupled to the pressure sensor, the adjustable component, the activity sensor, and the at least one pacing component. The processor may be configured to acquire activity data from the activity sensor to determine a level of activity of the patient; acquire pressure data from the blood pressure sensor to determine blood pressure adjacent the region; and adjust the adjustable component based at least in part on the determined blood pressure.

In accordance with another embodiment, a method is provided for monitoring and/or treating a heart of a patient to increase the patient's capacity for physical activity that includes acquiring activity data from an activity sensor implanted in the patient's body to determine a level of activity of the patient; acquiring pressure data from a blood pressure sensor implanted with a region of the heart to determine blood pressure adjacent the region; and adjusting an adjustable component to affect contractility of a right ventricle of the heart based at least in part on the determined blood pressure.

In accordance with yet another embodiment, a method is provided for monitoring and/or treating a patient to increase the patient's capacity for physical activity using an adjustable component disposed within a right ventricle of a heart of the patient that includes monitoring an activity level of the patient, blood pressure within the heart, and cardiac output of the heart using one or more sensors; determining a target cardiac output for the heart; if the actual cardiac output of the heart is greater than the target cardiac output, determining a target blood pressure for the patient; if the blood pressure is greater than the target blood pressure, adjusting the adjustable component within the right ventricle to reduce the blood pressure.

In accordance with still another embodiment, a method is provided for monitoring and/or adjusting an electrical system of a heart of a patient that includes acquiring signals from a sensing element within the heart indicating normal timing of activation of a right ventricle of the heart; and delivering one or more electrical signals to a free wall of the right ventricle before activation of the right ventricle to affect function of the right ventricle.

In accordance with another embodiment, a system is provided for monitoring and/or adjusting an electrical system of a heart of a patient that includes an elongate member sized for introduction into a right ventricle of the heart, the elongate member carrying a sensing element; one or more electrodes configured for introduction into the right ventricle; and a processing unit operatively coupled to the sensing element and one or more electrodes. The processing unit may be configured to acquire signals from the sensing element to determine normal timing of activation of the right ventricle; and deliver one or more electrical signals to a free wall of the right ventricle via the one or more electrodes before activation of the right ventricle to compromise normal function of the right ventricle.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It will be appreciated that the exemplary apparatus shown in the drawings are not necessarily drawn to scale, with emphasis instead being placed on illustrating the various aspects and features of the illustrated embodiments.

FIG. 1 shows an exemplary embodiment of a system, e.g., an implantable pressure regulator/pacemaker/defibrillator, implanted within a patient's body.

FIG. 2 is a functional block diagram of exemplary circuitry of the system of FIG. 1.

FIG. 3 shows an exemplary embodiment of a device configured to stimulate the base of the right ventricular free wall.

FIG. 4 is a flow chart showing an exemplary algorithm that may be used by a system implanted within a patient's heart to improve the patient's capacity for exercise, e.g., by decreasing right ventricular stroke volume through electrical stimulation.

FIG. 5 is a flow chart showing another exemplary algorithm that may be used by a system implanted within a patient's heart to improve the patient's capacity for exercise.

FIG. 6 is a flow chart showing yet another exemplary algorithm that may be used by a system implanted within a patient's heart to improve the patient's capacity for exercise.

### DETAILED DESCRIPTION OF THE EXEMPLARY EMBODIMENTS

In the following description, numerous details are set forth in order to provide a more thorough description of the system. It will be apparent, however, to one skilled in the art, that the disclosed system may be practiced without these specific details. In the other instances, well known features have not been described in detail so as not to unnecessarily obscure the system.

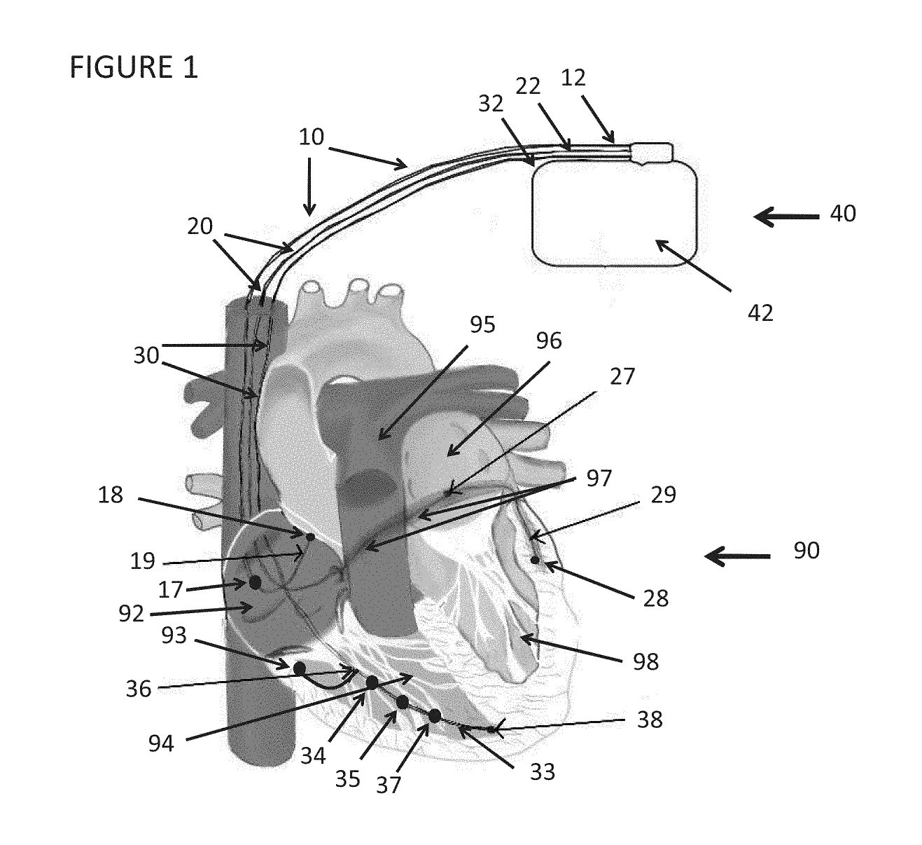

Turning to the drawings, FIG. 1 shows an exemplary embodiment of a system 8 including a pressure regulator/pacemaker/implantable cardio-defibrillator (ICD) with specialized leads implanted and/or introduced into a patient's heart 90, e.g., for practicing the exemplary systems and methods described elsewhere herein. In the embodiment shown in FIG. 1, the system 8 includes a controller 40 including a housing 42 sized and/or otherwise designed to be implanted within the patient's body, e.g., subcutaneously or within the thoracic cavity adjacent the heart 90. The housing 42 of the controller 40 is connected to several leads 10, 20, 30 that are designed to be implanted into the patient's heart 90.

For example, as shown, a first lead 10 may include a first or proximal end 12 coupled to the housing 42 and a distal or second end sized for introduction into the patient's heart 90, e.g., into the right atrium 92. The first lead 10 may have a distal end 19 carrying a sensor and/or electrode 18 for sensing electrical activity (depolarizations) and/or pacing the right atrium 92, as programmed, e.g., as described elsewhere herein. In addition, the distal end 19 of the first lead 10 may include one or more features, e.g., a screw tip or other anchor (not shown) on its distal tip for securing the distal end 19 relative to the wall of the right atrium 92 or left atrium 96. One or more wires or other conductors may extend from the distal end 16 to the proximal end 12 to communicate the signals from the sensor 18 to the controller 40.

Similarly, a second lead 20 may include a first or proximal end 22 coupled to the housing 42 and a second or distal end 29 sized for introduction into the patient's heart 90, e.g., into the right atrium 92 through the coronary sinus 97 or other vein of the heart 90. The second lead 20 may include a first sensor or electrode 27 carried on the distal end 29 designed to sense or measure pressure in the left atrium 96. In addition, the second lead 20 may include a second sensor or electrode 28 carried on the distal end 29, e.g., adjacent a distal tip of the second lead 20 and/or otherwise distal to the first sensor 27 for sensing or measuring pressure located within the distal coronary sinus 97, which may be reflective of left ventricle 98 pressures. Similar to the first lead 10, the second lead 20 may include one or more features, e.g., a screw tip or other anchor (not shown), on the distal tip to secure the distal end 29 within the patient's heart 90, e.g., within the coronary sinus 97, similar to pacing leads. Alternatively, the first and second leads may be provided on a single device with a branched distal end (not shown), similar to embodiments described in the applications incorporated by reference herein.

Additionally, a third lead 30 may be provided that includes a proximal or first end 32 coupled to the housing 42 and a distal or second end 33 sized for introduction into the patient's heart 90, e.g., into the right atrium 92, through the tricuspid valve 93 and into the right ventricle 94. The third lead 30 includes one or more sensors or electrodes on the distal end, e.g., a first electrode and/or sensor 38 designed to sense electrical activity or deliver electrical energy to stimulate the right ventricle 96.

Similar to the first lead 10 and second lead 20, the third lead 30 may include one or more features, e.g., a screw tip or other anchor (not shown), on the distal tip to secure the distal end within the patient's heart 90, e.g., into the wall of the right ventricle 94, similar to typically used pacing leads. The third lead 30 may sense and pace electrical activity occurring in the right ventricle 94.

In addition, the third lead 30 may include a plurality of additional electrodes spaced apart from one another along the distal end 33, e.g., electrodes 34 and 35 designed to sense electrical capacitance at several points in time throughout the cardiac cycle to estimate the stroke volume of the right ventricle 94. For example, changes in impedance throughout the cardiac cycle may be used to estimate volume changes in the right ventricle. Even with drift in electrical signals over time, these measurements may be used by the controller 40 to determine changes in volume which may be used to guide device functioning.

In addition, the third lead 30 may include an adjustable component 36 designed to affect the volume of blood that can fill the right ventricle 94, e.g., similar to the devices described in the applications incorporated by reference herein. This component 36 may communicate between the right atrium 92 and right ventricle 94. In one embodiment, this communication allows the component 36 to displace blood from the right ventricle 94. The component 36 may change shape and/or size, or simply move along the tricuspid annulus. In other embodiments, the component 36 may transfer fluid or gas between two chambers to alter stroke volume.

In another embodiment, the component 36 may be entirely situated within the right ventricle 94 and/or make the flow of blood into the pulmonary artery 95 more difficult. In this manner, the right ventricular stroke volume may be decreased to optimize left-sided and/or pulmonary pressures. In still another embodiment, the component 36 may enlarge and/or travel along the lead 30, e.g., in order to displace blood within the right ventricle 94. In another embodiment, the controller 40 may selectively enlarge a balloon within the right ventricle 94 instead of the component 36.

One or more pressure sensors may be placed at desired locations, e.g., in the left atrium, on the interatrial septum, or in the coronary sinus 97 (with occlusion to optimize pressure recordings), e.g., on the lead 20 and/or separate leads (not shown) in order to estimate left-sided filling pressures. In addition or alternatively, one or more pressure sensors may be placed in the right atrium 92, right ventricle 94, right ventricular outflow track, or pulmonary artery 95, e.g., one leads 10, 30, and/or separate leads (not shown). By combining flow measurements with pressure sensors within the blood system prior to the pulmonary vasculature, filling pressures from the left atrium and/or left ventricle 98 may be estimated. The waveform analysis may include absolute pressures and/or the slope or change in pressure (tao) during the cardiac cycle.

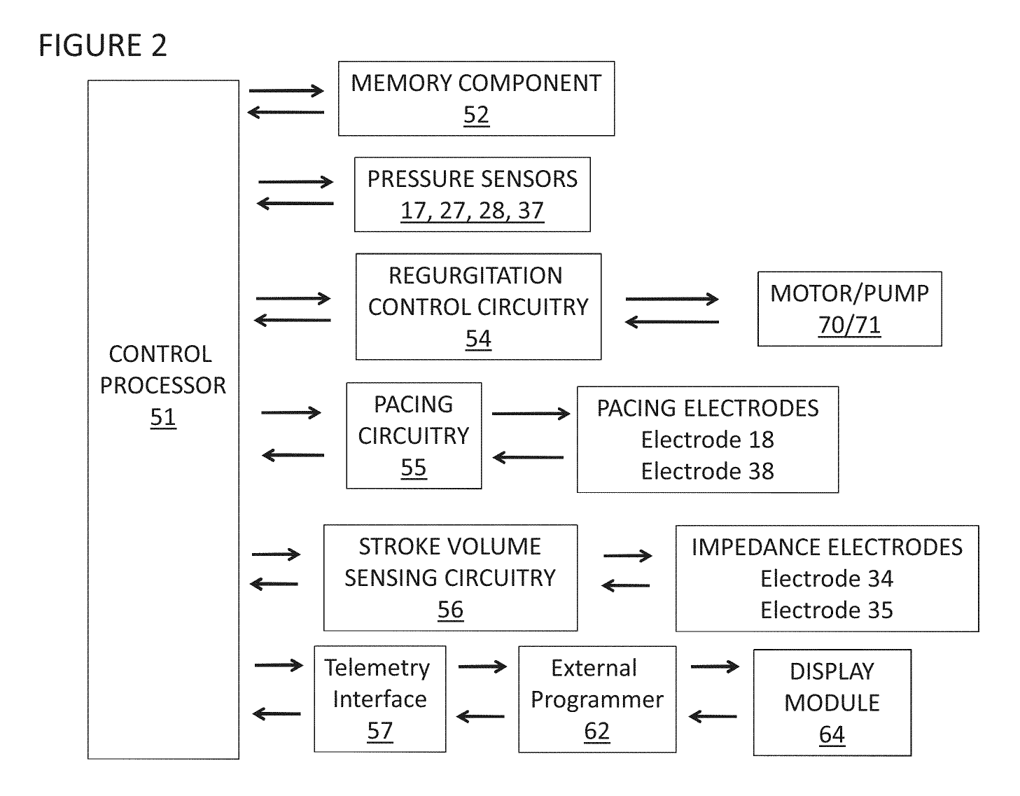

FIG. 2 shows a simplified functional block diagram of one embodiment of the components located within and/or connected to the controller 40. In the embodiment shown, the components include a control processor 51, which receives input information from various components, e.g., sensors, electrodes, and/or other components on the leads 10, 20, 30, in order to determine the function of the different components to treat the patient. For example, the control processor 51 is connected to a memory component 52 provided within the housing 42, pressure sensors (e.g., sensors 17, 27, 28, and 37 provided on the leads 10, 20, 30), pacing circuitry 55, stroke volume sensing circuitry 56, and a telemetry interface 57, e.g., provided within the housing 42. The pacing circuitry 55 connects to the electrodes, for example, electrodes 18 and 38. These connections allow for multiple capacities to sense electrical activity (such as myocardial depolarizations), deliver pacing stimulations, and/or deliver defibrillation or cardioversion shocks.

The stroke volume sensing circuitry is connected to electrodes, for example electrodes 34 and 35. These electrodes sense change in impedance values at periods of the cardiac cycle to estimate stroke volume. Optionally, the control processor 51 is connected to a telemetry interface 57. The telemetry interface may wirelessly send and receive data from an external programmer 62 which is coupled to a display module 64 in order to facilitate communication between the control processor 51 and other aspects of the system external to the patient.

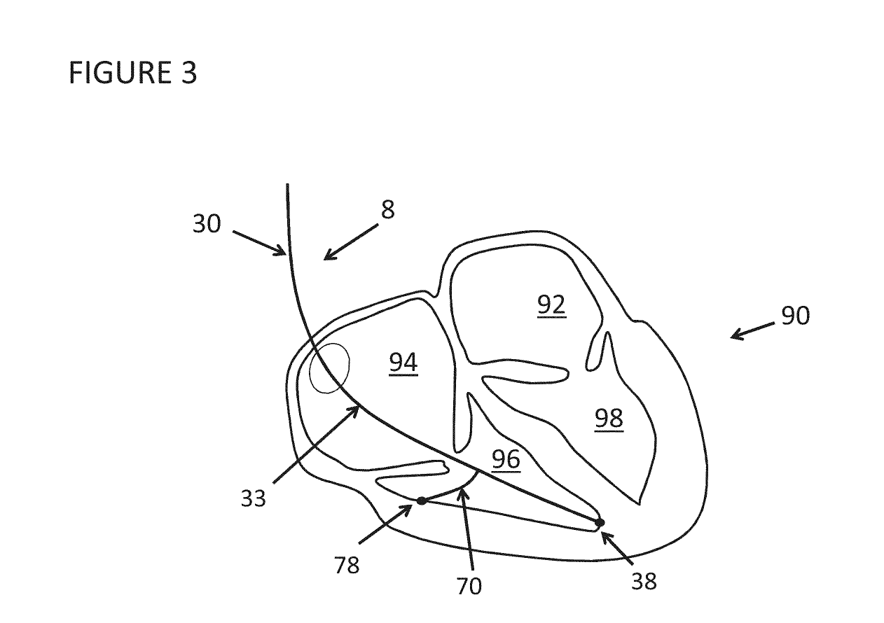

FIG. 3 shows another exemplary embodiment of a system 8 including one or more specialized leads implanted and/or introduced into a patient's heart 90, e.g., for practicing the exemplary systems and methods described elsewhere herein. For example, the system 8 may include a lead 30 including a proximal end coupled to a controller (not shown) outside the heart 90 and a distal end 33 sized for introduction into one or more chambers of the heart 90. As shown in FIG. 3, a distal tip of the lead 30 is coupled to the apex of the right ventricle 96, e.g., by an electrode 38. Optionally, the electrode 38 and/or distal tip of the lead 30 may include a screw tip or other anchor (not shown) for securing the distal tip and/or electrode 38 to the apex.

In addition, the distal end 33 of the lead 30 includes a branch member or lead 70 that extends transversely from the distal end 33 proximal to the distal tip. The branch member 70 includes one or more electrodes, e.g., right ventricular free wall electrode 78, configured to sense and stimulate the myocardium located at the base of the right ventricle 96. The electrode 78 may be configured to be placed along the free wall of the right ventricle, e.g., at a tip electrode on a distal tip of the branch 70 that may be positioned against the wall and optionally secured thereto, e.g., using a screw tip or other anchor (not shown). Alternatively, the electrode 78 may be a ring or other electrode on a side wall (not shown) of the branch member 70 that may be placed against the free wall. For example, the branch member 70 may be biased to a predetermined shape and/or have a minimal length to place the branch 70 along the free wall such that the electrode 78 sufficiently contacts the wall.

In an exemplary embodiment, the branch member 70 may be biased to naturally extend away from the distal end 33 at a predetermined angle yet be deflectable to accommodate introduction. For example, at least a portion of the branch member 70 may include a spring mechanism or may be made of material designed to extend away from the distal end 33 to maintain a predetermined force against the free wall, e.g., to place the electrode 78 sufficiently in contact with the free wall. Alternatively, the electrode 78 may also be implanted separate from the lead 30 and communicate to the processor through wireless transmission.

In yet another embodiment, the right ventricular free wall electrode may be provided on an entirely separate secondary lead or elongate member (not shown) including a proximal end coupled to the controller and distal end including a screw tip or other anchor (also not shown) such that a distal tip of the secondary lead may be screwed into and/or otherwise secured to the free wall to place the electrode 78 into contact therewith. Optionally, the system 8 may also include a delivery sheath, catheter, and/or other delivery devices configured to deliver the electrode 78 into the desired location.

In still another embodiment, a plurality of electrodes may be provided on the branch member 70 (or separate secondary lead) that are spaced apart from one another. In this embodiment, the branch member 70 (or separate secondary lead) may have sufficient length to place the electrodes against the free wall of the right ventricle, e.g., from the base partially towards the apex.

A processor within the controller may communicate with the electrodes 38, 78 to determine if and when the base of the right ventricle 96 should be stimulated. In the setting of increased patient activity or rising/elevated pulmonary pressures (or any sensor that suggests elevated left ventricular 98 filling pressures), the processor may deliver electrical signals, e.g., via electrode 78 (or a plurality of electrodes along the free wall) that result in premature activation of the right ventricle 96, i.e., before the rest of the ventricular myocardium. In this way, the stroke volume of the right ventricle 96 may be decreased in order to optimize intracardiac hemodynamics.

Optionally, the processor may adjust timing of stimulation provided by the right ventricular free wall electrode 78 depending on the amount of sensed patient activity based at least in part on sensed blood pressure, e.g., using one or more sensors on the lead 30 (or another lead positioned elsewhere within the heart 90, similar to other embodiments herein). In this way, the effect on right ventricular function may be titrated based on the clinical circumstance. Specifically, more premature pacing may be required to affect right ventricular function more dramatically, while in other circumstances only mild premature pacing may be required. In addition, a blood pressure sensor may be placed within the right ventricle 96 or other chamber (such as the pulmonary artery, not shown) to monitor the blood pressure within the respective chamber or vessel.

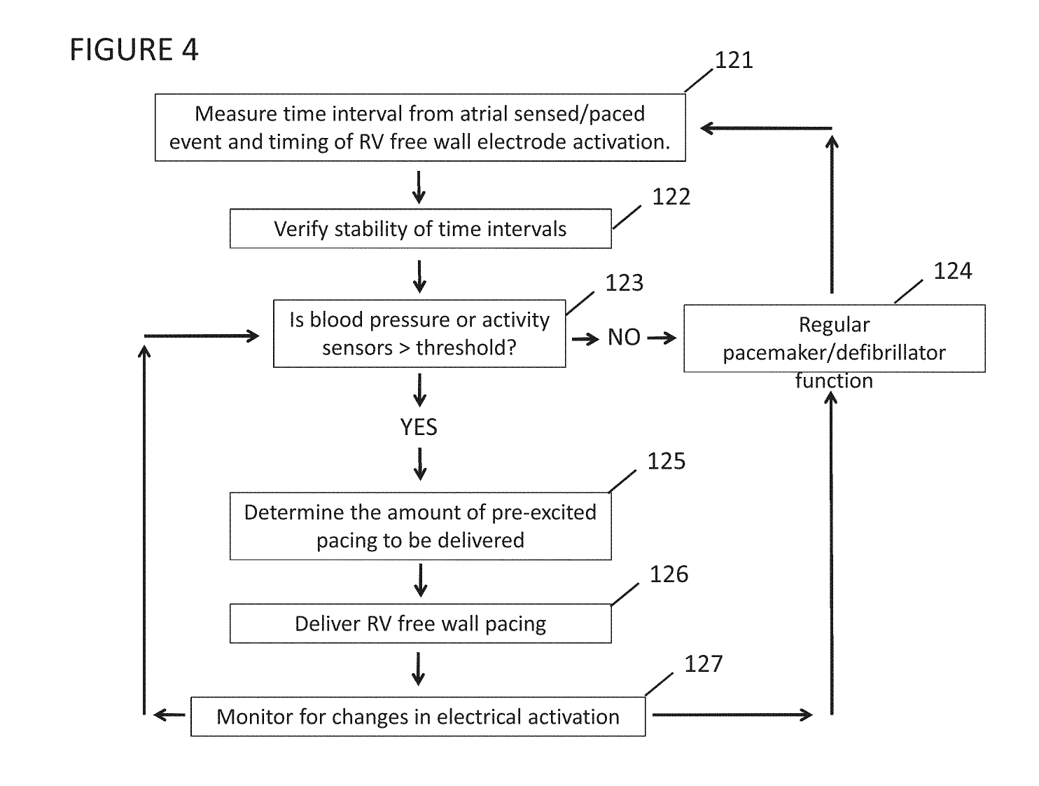

FIG. 4 is a flow diagram showing an exemplary algorithm that may be used by a system, such as those shown in FIGS. 1-3 including a processor communicating with one or more sensors within the right ventricle of a patient's heart, to improve the patient's capacity for exercise. In step 121, the processor measures the time interval from the atrial sensed or paced event and the subsequent timing of the right ventricular free wall electrode activation. Optionally, the processor may also determine the timing of right ventricular apex through additional electrodes or far-field waveform analyses. In step 122, the processor verifies whether the timing of the intervals is stable and predictable.

Turning to step 123, additional sensors determine if a sensed blood pressure or patient activity exceeds a determined threshold. If the sensed parameter is not greater than the set threshold, the system moves to step 124 where the system functions as a typical pacemaker or cardio-defibrillator. The system then continues back to step 121 and continues to measure and monitor the time intervals in step 121. If, however, the measured parameters are greater than the set threshold, the system moves to step 125. Here, the amount of premature RV free wall pacing is determined. The amount of premature pacing may depend upon the sensed blood pressure or patient activity. For example, if sensed blood pressure is only mildly elevated beyond the set threshold, the prematurity of the right ventricular free wall electrode 78 may be minimal. Alternatively, if the pressure is severely elevated (or rises rapidly), the prematurity of the pacing may be increased.

Moving to step 126, after determining the amount of premature pacing, the processor activates one or more electrodes, such as electrode 78 in FIG. 3, to deliver premature pacing to the right ventricular free wall (or other location within the right ventricle in efforts to influence the activation of right ventricular contraction). Moving to step 127, the processor may then monitor for changes in electrical activation. This may include far-field morphology analyses or additional electrodes. For example, the system may include an electrode configured and/or positioned to sense left ventricular activation or right ventricular apex activation. If ventricular cardiac ectopy or other abnormality is sensed, the processor may move to step 124 to stop premature pacing and deliver standard pacemaker or defibrillator therapy. If no abnormality is detected, the processor may move to step 123 in a closed loop system to continually adjust the prematurity of RV pacing based on sensed activity.

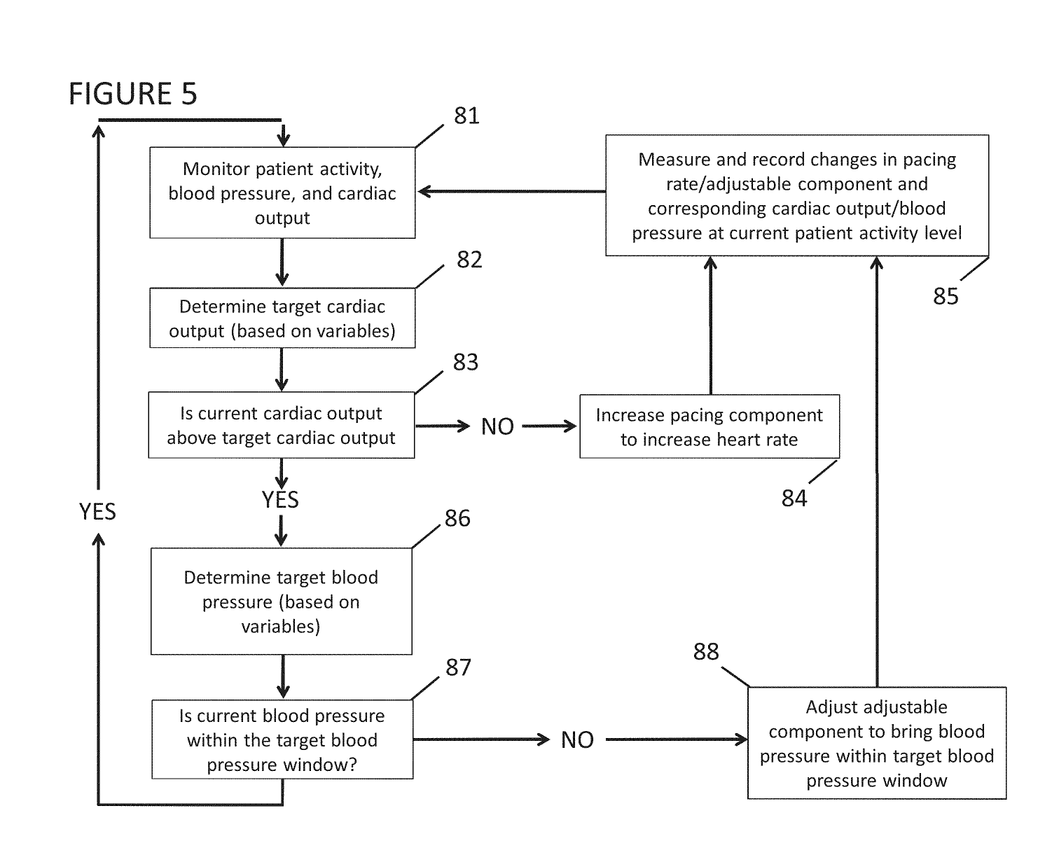

Turning to FIG. 5, another exemplary embodiment of an algorithm is shown that may be used by a system, such as that shown in FIGS. 1 and 2 including a processor communicating with one or more sensors and an adjustable component within the right ventricle of a patient's heart, to improve the patient's capacity for physical activity. In this embodiment, the system may include one or more sensors coupled to the processor for providing data indicative of patient activity, blood pressure, and/or cardiac output into a closed loop system. In step 81, the processor monitors patient activity, blood pressure, and cardiac output. In step 82, the processor determines the target cardiac output. This output may be based on patient activity sensors and sensors corresponding to total body volume status. For example, if the blood pressure sensors record elevated pressures, the processor may be programmed to increase cardiac output in order to encourage renal perfusion and subsequent diuresis.

Moving to step 83, the processor determines if the measured cardiac output is greater than the target cardiac output. If the cardiac output is not above target output, the processor moves to step 84, where the heart rate is increased to improve cardiac output. The processor then moves to step 85 to measure and record changes in sensed parameters to tracked relationships between sensed patient activity, blood pressure, heart rate, and the adjustable component. In exemplary embodiments, the adjustable component may located and/or configured to affect tricuspid regurgitation, cause premature pacing of the right ventricular free wall, or cause a pressure gradient within the vena cava, e.g., similar to systems described in the applications incorporated by reference herein.

Moving to step 86, in the setting that the cardiac output is above the target cardiac output, the processor determines a target blood pressure that sets a threshold for activation of the adjustable component. As long as the sensed blood pressure remains below the target blood pressure, the processor may leave components of the system inactive without initiating treating the patient. For example, during increased patient activity, the processor may increase the target blood pressure to accommodate the increased activity. This is because the system is attempting to prevent pressure overload within the heart. However, higher filling pressures may improve forward flow. The processor may later identify the relationship between filling pressures and cardiac output. The slope of this relationship may be used to determine the target blood pressure.

Then, moving to step 87, the processor determines if the measured blood pressure is below the target blood pressure. If the pressure is too high, for example, the processor moves to step 88, where the adjustable component is activated to lower the blood pressure. This adjustable component may include a mechanism to affect tricuspid regurgitation, premature pacing of the right ventricular free wall, or the creation of a pressure gradient within the vena cava, e.g., similar to the embodiments described elsewhere herein and in the applications incorporated by reference herein. The processor then moves to step 85 where the relationships between the various components are recorded and analyzed to further optimize the system. If, in step 87, the blood pressure is within the target window, the processor moves back to step 81 to continue monitoring patient activity, blood pressure, and cardiac output.

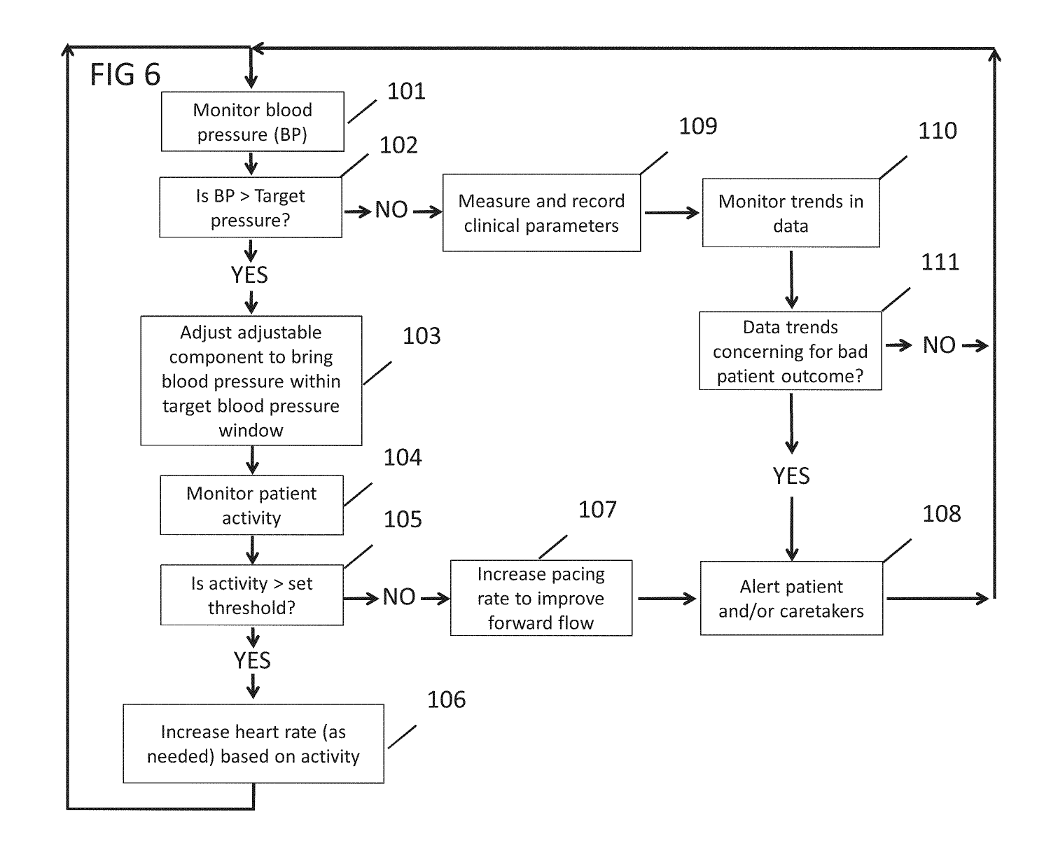

Turning to FIG. 6, yet another exemplary embodiment of an algorithm is shown that may be used by a system, such as that shown in FIGS. 1-3 including a processor communicating with one or more sensors and an adjustable component within the right ventricle of a patient's heart, to improve the patient's capacity for physical activity that does not require cardiac output monitoring. In this embodiment, the processor starts by measuring a blood pressure, in step 101. This blood pressure may refer to central venous pressure, right atrial pressure, right ventricular pressure, pressure within the coronary sinus, pulmonary artery pressure, or left atrial pressure. Moving to step 102, the processor determines if the measured pressure is above the set target blood pressure. If the pressure is not above the target blood pressure, the processor moves to step 109, where clinical parameters are recorded and tracked. Moving to step 110, the parameters are tracked and trends in data are monitored.

Moving to step 111, if the trends in the data indicate a bad outcome of the patient, the processor moves to step 108 where the patient and/or caretakers/clinicians are made aware of the data trends. If the blood pressure is elevated in step 102, the processor moves to step 103, where the adjustable component is activated to reduce the blood pressure. Again, the adjustable component may include a mechanism to affect tricuspid regurgitation, premature pacing of the right ventricular free wall, or the creation of a pressure gradient within the vena cava, as described elsewhere herein and in the applications incorporated by reference.

Moving to step 104, the processor measures the patient activity. If the patient activity is above a set threshold in step 105, the processor moves to step 106 where the heart rate is determined based on the sensed activity level. The processor may then move back to step 101 to continue to monitor the blood pressure. If the sensed activity level is not elevated in step 105, the processor moves to step 107, where the pacing rate is increased to encourage cardiac output/forward flow. By increasing the heart rate, the processor may attempt to encourage renal perfusion to encourage diuresis to reduce total body blood volume. In addition to increasing the pacing rate in step 107, the processor may move to step 108, where an alert is sent to the patient or caretakers/clinicians. This alert may indicate that medications need to be changed or other action needs to be taken to help reduce filling pressures. The processor then moves back to step 101 and continues to monitor the blood pressure in a closed loop system. The goals of this closed-loop system may be to optimize the blood pressure and heart rate to optimize patient performance, reduce heart failure admissions, and/or alert patients and clinicians of potential bad outcomes, such as a heart failure admission.

In accordance with another embodiment, systems and methods are provided for determining optimal volume status and/or filling pressures for a patient's heart, e.g., depending on the patient's level of activity. Optimal left-sided filling pressures may vary depending on activity level and body positioning. Furthermore, just because an increase in left-sided filling may improve cardiac output does not intrinsically mean left-sided tilling pressures should be increased. In fact, depending on the patient's sensitivity to elevated right and left-sided filling pressures, the optimal volume status and filling pressures may not be clear from hemodynamic data alone. Therefore, in some cases, sensors of respiratory data and/or patient symptoms may need to be incorporated into the system to identify optimal blood filling pressures.

In one embodiment, a system may be provided that includes one or more sensors corresponding to respirations (both tidal volumes and respirator rate) and/or activity levels while the system simultaneously adjusts left-sided filling pressures. Over time, the system may identify the blood pressure and/or acute change in blood pressure at which respiratory rate exceed the oxygen demands of the body. This point may theoretically represent the respiratory threshold. The system may monitor this pressure (or pressure change) to identify when the patient reaches this threshold or to monitor improvement in breathing patterns based on device action.

The system may also be used as a treatment for sleep apnea. Elevations in sleep apnea may be caused by elevated blood pressures. The system may monitor and treat elevated lung/heart pressures to prevent sleep apnea. In addition, the system may sense sleep apnea through changes in respiratory sensors and reduce blood pressures empirically.

Some of the systems and methods described herein may include a pressure sensor to monitor one or more blood pressures near the heart. These pressures may reflect left-sided filling pressures, right-sided filling pressures, systemic pressure, pressure within the coronary sinus, and/or pulmonary pressures. For example, the pressure sensor may be placed in the inferior vena cava, right ventricle, pulmonary artery, left atrium, and/or coronary sinus. In some embodiments the system may measure changes in impedance or mechanical deflections in order to identify changes in a blood pressure.

Heart failure is a chronic disease that is difficult to monitor. Any reduction in left-sided filling pressures by a system implanted in the patient's heart may result in a reduction in natriuretic peptides, such as brain natriuretic peptide (BNP). If a patient is volume overloaded at baseline and the system reduces left-sided filling pressure, the reduction in pressure may drop natriuretic peptides and result in the kidneys increasing salt and water resorption to increase total body volume. Therefore, in some cases, total body blood volume may be best managed by medications. In these cases, the system may send pressure information to help guide the patient and his/her clinicians to optimize medical management. In addition, reducing left-sided filling pressures may actually improve forward flow (by reducing mitral valve regurgitation, for example).

In other circumstances, increasing the heart rate can increase forward flow. However, increasing heart rate without adjusting the balance of bi-ventricular function often increases pulmonary pressures and therefore worsens patient respiratory status and/or patient quality of life. In some cases, it may be advantageous to increase heart rate and optimize left-sided filling pressures to increase forward flow. By increasing forward blood flow, diuresis may be encouraged. Therefore, systems and methods may be provided for optimizing left-sided filling pressures. However, if there is evidence that left-sided filling pressures are elevated at baseline (when the patient is not exerting themselves), reducing left-sided filling pressure without encouraging forward flow may exacerbate volume status.

Therefore, in one embodiment, if there is evidence of increased total body blood volume (for example through impedance measurements, pressure measurements, or blood flow measurements), the system may increase the heart rate to increase forward flow. By increasing the heart rate, the cardiac output may increase. The higher cardiac output may facilitate the kidneys to diuress more fluid. Therefore, when the patient is volume overloaded, the device may increase the heart rate in order to reduce total body blood volume.

In other embodiments, the system may increase heart rate proportion to the amount of overload in total body blood volume. While the system increases heart rate to encourage forward flow, the system may also adjust biventricular function (e.g., by inducing tricuspid regurgitation, creation of a pressure gradient within the heart or vena cava, or pacing the right ventricular free wall prematurely, as described elsewhere herein) to prevent the left-sided filling pressures from becoming pressure overloaded (or to reduce left-sided filling pressures). Therefore, the patient's heart rate may slowly increase over time if the patient is non-compliant with their medications.

In some instances, this therapy may preclude the requirement of medications to prevent volume overload. In other cases, medications are still required. Furthermore, while the system may optimize patient symptoms, left-sided filling pressures, and cardiac output, the system may also send trends in patient activity, volume status, amount of required regurgitation, heart rate, and other variables to alert clinicians of potential interventions needed to prevent a heart failure admission. Combinations in variables may be identified to calculate risk of heart failure admissions and alert caretakers to facilitate patient management and prevent heart failure admissions.

In one embodiment, the system includes an adjustable component capable of inducing blood regurgitation across a valve, such as the tricuspid valve. The adjustable component may be positioned along an elongate member. In one embodiment, an elongate member connects the processor to a pacing electrode in the heart; along the course of the body of the elongate member, an adjustable component is capable of inducing blood flow regurgitation across the tricuspid valve. In another embodiment, a pressure sensor may be positioned along the elongate member that measures a blood pressure. In another embodiment, the pressure sensor, pacing electrode, and adjustable component are all positioned on the same elongate member. In another embodiment, the pressure sensor, pacing electrode, and adjustable component are located on separate elongated members, or are wireless devices but working in combination.

The system may also include pacemaker functions typical of most pacemaker systems. Furthermore, the system may include anti-tachycardia pacing (ATP) and defibrillation capabilities typical of most implantable cardioverter-defibrillator (ICU) devices. The system may include activity sensors, such as accelerometer that is indicative of patient movement. Other sensors may be indicative of patient position, such as lying down. In another embodiment, the system includes sensors that correspond to minute ventilation or respiratory rate.

The target left-sided filling pressure may be determined by trends in the data or may be programmed by the patient's clinicians. For example, the first step in optimizing left-sided filling pressures may include controlling tricuspid regurgitation to balance bi-ventricular function. However, if the left-sided filling pressures remain elevated, the heart rate may be increased to encourage forward flow. In one embodiment, the system includes flow sensors that monitor cardiac output or stroke volume. The heart rate may be increased while optimizing left-sided filling pressures.

In some embodiments, the relationship between heart rate, blood pressure, and cardiac output are monitored in order to guide the system to optimize the heart rate and filling pressures. In other embodiments, the clinicians may program the response of the heart rate to elevated sensors of volume status. For example, the programmed heart rate may correspond to the amount of regurgitation required to maintain left-sided filling pressures or to other markers of blood volume (such as thoracic impedance measurements, and blood pressure trends).

In another embodiment, the system increases the heart rate of the patient based at least in part on data from activity sensors and/or heart failure status. Heart failure status may be determined by impedance measures, required tricuspid regurgitation to maintain the blood pressure, a blood pressure, or patient input/responses. Activity level may be determined by accelerometers or respiratory sensors. Therefore, the programmed heart rate may be determined by activity sensors and heart failure or volume status sensors. For example, if a patient is non-compliant, the patient's volume status and/or other marker of volume status (such as pulmonary pressure, left atrial pressure, right atrial pressure, impedance markers, etc.) may continue to rise. As the heart failure is getting worse and total volume status increases, the heart rate may be increased to encourage forward flow and diuresis. In some cases, increase in cardiac output alone may be enough to encourage diuresis and prevent volume overload.

In some embodiments, the device monitors stroke volume and/or cardiac output. Stroke volume may be estimated by measuring oxygen saturation of the blood (e.g., using light spectroscopy), impedance changes, thermodilution, flow sensors, or changes in pressure. However, the system may also increase heart rate without directly monitoring cardiac output.

Therefore, there are numerous functions of the described system. First, during activity or exertion, the system induces tricuspid regurgitation to prevent left-sided filling pressures from becoming pressure or volume overloaded. Furthermore, the system may also increase the heart rate plus or minus controlling left-sided filling pressures (for example through regurgitation), in order to increase cardiac output and encourage diuresis without increasing left-sided filling pressures. Therefore, the system may increase exercise capacity and quality of life while simultaneously prevent volume overload by increasing cardiac output. Furthermore, the system may contact the patient and/or clinicians to alert of changes in volume status, pressure trends, or other sensor data. These alerts can facilitate action to start new medications, increase medication dose, or other interventions to prevent a heart failure admission and/or other deterioration in clinical or heart function.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A system configured to be implanted in a patient's body to monitor and/or adjust electrical activation of the patient's heart, the system comprising:
at least one sensor acquiring signals corresponding to patient activity or movement;
at least one pacing component positioned adjacent a free wall of a right ventricle of the heart, whereby stimulation of the at least one pacing component is designed to compromise function of the right ventricle; and
a controller coupled to the at least one sensor and at least one pacing component for adjusting the function of the at least one pacing component based at least in part on the signals from the at least one sensor.

2. The system of claim 1, further comprising an additional sensor measuring sensor data corresponding to a blood pressure.

3. A system for monitoring and/or treating a heart of a patient to increase the patient's capacity for physical activity, comprising:
a blood pressure sensor implantable within a region of the heart;
an adjustable component configured to affect contractility of a right ventricle of the heart;
an activity sensor implantable within the patient's body;
at least one pacing component sized for introduction into a region of the heart; and
a processor operatively coupled to the pressure sensor, the adjustable component, the activity sensor, and the at least one pacing component to:
acquire activity data from the activity sensor to determine a level of activity of the patient;
acquire pressure data from the blood pressure sensor o determine blood pressure adjacent the region; and
adjust the adjustable component based at least n part on the determined blood pressure.

4. The system of claim 3, wherein the processor is configured to estimate the total body blood volume based at least in part on the activity data and pressure data.

5. The system of claim 4, wherein the processor is further configured to increase a pacing rate to the pacing component based at least in part on the estimated total body blood volume.

6-10. (canceled)

11. A method for monitoring and/or adjusting an electrical system of a heart of a patient, the method comprising:
acquiring signals from a sensing element within the heart indicating norm al timing of activation of a right ventricle of the heart; and
delivering one or more electrical signals to a free wall of the right ventricle before activation of the right ventricle to affect function of the right ventricle.

12. The method of claim 11, wherein the sensing element is positioned within the right ventricle.

13. The method of claim 11, wherein the sensing element is positioned at an apex of the heart within the right ventricle to detect electrical signals from the heart identifying activation of the right ventricle.

14. The method of claim 11, wherein the one or more electrical signals are delivered to the free wall via one or more electrodes positioned against the free wall.

15. The method of claim 14, wherein the one or more electrodes comprises a first electrode positioned at a base of the right ventricle.

16. The method of claim 15, wherein the one or more electrodes further comprise a second electrode spaced apart from the first electrode between the first electrode and an apex of the heart, and wherein delivering one or more electrical signals comprises sequentially delivering one or more electrical signals to the first and second electrodes.

17. The method of claim 11, further comprising monitoring pressure within one or more regions of the heart to determine when there is an increase in the pressure; and wherein the one or more electrical signals are delivered to compromise the function of the right ventricle in order to reduce the pressure.

18-19. (canceled)

20. The method of claim 11, wherein the one more electrical signals are delivered to temporarily decrease the right ventricular contractility.

21. A system for monitoring and/or adjusting electrical activation of a heart of a patient, the system comprising:
an elongate member sized for introduction into a right ventricle of the heart, the elongate member carrying a sensing element;
one or more electrodes configured for introduction into the right ventricle; and
a processing unit operatively coupled to the sensing element and one or electrodes to:
acquire signals from the sensing element to determine normal timing of activation of the right ventricle; and
deliver one or more electrical signals to a free wall of the right ventricle via the one or more electrodes before activation of the right ventricle to compromise normal function of the right ventricle.

22. The system of claim 21, further comprising a sensor sized for introduction into the heart for providing signals corresponding to pressure within a region of the heart, wherein the processing unit is configured to monitor pressure within the region of the heart to determine an increase in the pressure, and wherein the processing unit delivers the one or more electrical signals to the free wall in response to the increase in pressure.

23. The system of claim 21, wherein:
the elongate member comprises a lead including a distal end sized for introduction into the right ventricle, wherein the sensing element is carried on the distal end, and
wherein the lead further comprises a branch member extending transversely from the distal end at a location proximal to the sensing element, the branch member carrying the one or more electrodes.

24-27. (canceled)

28. The system of claim 21, further comprising an implantable electrode device including the one or more electrodes, the electrode device including a housing configured for implantation within the right ventricle and a wireless communication interface for communicating with the processing unit to receive instructions to activate the one of m ore electrodes to deliver the one or more electrical signals to the free wall.

29. (canceled)

30. The system of claim 21, further comprising a motion sensor for measuring movement of the patient, the processing unit coupled to the motion sensor to determine when an increase in the movement indicates an increased level of activity of the patient, and wherein the processing unit delivers the one or snore electrical signals to the free wall when the increase in movement indicates an increased level of activity requiring a reduction in normal function of the right ventricle.

31-42. (canceled)
